(19) United States
(12) Patent Application Publication
(10) Pub. No.: US 2010/0016675 A1
(43) Pub. Date: Jan. 21, 2010

(54) Method of assessing a condition using sucking patterns

(76) Inventor: Jason C. Cohen, Appleton, WI (US)

Correspondence Address: KIMBERLY-CLARK WORLDWIDE, INC.;Tara Pohlkotte, 401 NORTH LAKE STREET, NEENAH, WI, 54956, US

(21) Appl. No.: 12/218,935

(22) Filed: Jul. 18, 2008

(51) Int. Cl. A61B 5/00

(52) U.S. Cl. 600/300

(57) ABSTRACT

A method of assessing a condition includes comparing a sucking pattern for a target infant with a sucking pattern associated with the condition. A method of monitoring progress of a treatment includes comparing the rhythmic sucking percentage for a target infant before treatment for a condition with the rhythmic sucking percentage for the infant after treatment of the condition to assess the effectiveness of the treatment. A method of monitoring progress of a treatment includes using sucking patterns to compare an infant's rate of progress at a given point in time to a population's rate of progress at the same point in time.

### BACKGROUND OF THE INVENTION

The comfort, well-being, or stress level of an infant in relation to their environment may be difficult to determine and/or quantify. Generally, an infant provides signals as to his or her comfort, well-being, or stress level in the form of facial expressions, other physical motions, and sounds. Some signals, such as smiling and cooing, may indicate that the infant is satisfied or happy with current environmental conditions and/or bodily conditions. Other signals, such as crying, generally indicate that the infant is not satisfied with current environmental conditions and/or bodily conditions. Such signals, whether indicative of a positive state of well-being (smiling and cooing) or a negative state of well-being (crying) may not be quantified and/or readily correlated to the infant's perception of their environment and may not be present in very young infants. This is particularly evident in infants having certain conditions such as colic. Colic is a condition in which an infant cries or screams for prolonged periods of time yet has no known health problems. As such, colic is difficult to diagnose with confidence. There are various treatments for colic including various herbal remedies, soothing measures, and dietary changes. However, improvement after treatment may be difficult to detect and monitor. Typically, a baby's level of colic is measured in hours of crying. This requires extensive monitoring and long durations of crying. Therefore, a method is needed to assist in determining the presence of various conditions such as colic. Additionally, a method is needed to evaluate the progress of an infant being treated for said conditions.

### SUMMARY OF THE INVENTION

In response to the needs discussed above, the present invention utilizes sucking patterns to assess various conditions. In one aspect, the present invention provides a method of assessing a condition in a target infant. The method includes the steps of: acquiring a first sucking pattern associated with the condition; acquiring a second sucking pattern for the target infant; comparing the second sucking pattern with the first sucking pattern; and determining if the second sucking pattern is consistent with the first sucking pattern.

In some embodiments, the first sucking pattern may be an aggregate pattern derived from a population of infants expressing the condition. In some embodiments, the second sucking pattern may be an aggregate pattern derived from the target infant on two or more occasions. In some embodiments, the first and/or the second sucking pattern may be acquired with a pacifier adapted to measure the intensity, frequency, pauses, number of sucks per envelope, and/or envelope function of the sucking patterns.

In some embodiments the second sucking pattern may be acquired in the target infant's residence and the comparing step and the determining step may be performed by a computer adapted to receive data from the pacifier. In some embodiments, the second sucking pattern may be acquired in a clinic and the comparison may be performed in a clinic. In some embodiments, the second sucking pattern may be acquired in a first location and the comparing step may be performed at a second location different than the first location. In some embodiments, the first location may be the infant's residence and the second location may be a clinic.

In some embodiments, the condition may be colic or cognitive alertness. In some embodiments, the determining step may be a subjective evaluation performed by a trained technician. In some embodiments, the determining step may be an objective evaluation based, at least in part, on pattern analyzing software, artificial intelligence, or neural networks.

In another aspect, the present invention provides a method of monitoring progress of a treatment of a target infant having a condition. The method includes the steps of acquiring a first sucking pattern for the target infant over a first period of time; calculating a first rhythmic sucking pattern percentage for the first period of time; acquiring a second sucking pattern for the target infant over a second period of time after the treatment of the target infant for the condition; calculating a second rhythmic sucking pattern percentage for the second period of time; comparing the first rhythmic sucking pattern percentage to the second rhythmic sucking pattern percentage; and assessing the effectiveness of the treatment.

In some embodiments, the method further includes the steps of: acquiring a third sucking pattern for the target infant over a third period of time after a second treatment of the target infant for the condition, wherein the second treatment is different than the first treatment; calculating a third rhythmic sucking pattern percentage for the third period of time; comparing the third rhythmic sucking pattern percentage to the second rhythmic sucking pattern or the first rhythmic sucking pattern; and determining the effectiveness of the first treatment relative to the second treatment.

In various embodiments, the first sucking pattern, the second sucking pattern, or the third sucking pattern may be electronically stored and/or may be electronically transmitted to a medical professional.

In some embodiments, the condition may be colic or cognitive alertness.

In another aspect, the present invention provides a method of monitoring progress of a treatment of a target infant having a condition. The method includes the steps of: acquiring a first sucking pattern for the target infant over a first period of time; calculating a first rhythmic sucking pattern percentage for the first period of time; acquiring a second sucking pattern for the target infant over a second period of time after a first treatment of the target infant for the condition; calculating a second rhythmic sucking pattern percentage for the second period of time; calculating a first rate of progress for the target infant at a first point in time; acquiring a second rate of progress as a function of time from a population having the condition and the treatment; comparing the first rate of progress to the second rate of progress at the first point in time; and assessing the progress of the first infant relative to the population at the first point in time.

In various embodiments, the second rate of progress may be an aggregate rate of progress as a function of time from a population of infants known to have the condition and the treatment. In various embodiments, the condition may be colic or cognitive alertness.

In some embodiments, the first sucking pattern may be acquired with a pacifier adapted to measure intensity, frequency, pauses, number of sucks per envelope, and/or envelope function of the first sucking pattern.

In some embodiments, the comparing or assessing steps may be a subjective evaluation performed by a trained technician, an objective evaluation based in part on pattern analyzing software, or combinations thereof.

### BRIEF DESCRIPTION OF THE DRAWINGS

FIG. 1 representatively illustrates a pacifier adapted to detect non-nutritive sucking events produced by a baby sucking on the pacifier.

FIGS. 2A, 2B, 2C, 2D, 2E, and 2F representatively depict modification of a pacifier into one version of a device adapted to detect non-nutritive sucking events and/or rhythmic sucking patterns.

FIG. 3 representatively depicts modification of a pacifier into one version of a device adapted to detect non-nutritive sucking events and/or rhythmic sucking patterns.

FIGS. 4A and 4B representatively depict modification of a pacifier into one version of a device adapted to detect non-nutritive sucking events and/or rhythmic sucking patterns.

FIGS. 5A and 5B representatively depict another version of a device adapted to detect non-nutritive sucking events and/or rhythmic sucking patterns.

FIG. 6 representatively depicts the interconnected components of one version of a device adapted to detect non-nutritive sucking events and/or rhythmic sucking patterns.

FIG. 7 representatively illustrates an exemplary rhythmic sucking pattern.

FIG. 8 representatively illustrates an exemplary non-rhythmic sucking pattern.

FIG. 9 representatively illustrates an exemplary rhythmic sucking pattern.

FIG. 10 representatively illustrates a flow diagram of one method of the present invention.

FIG. 11 representatively illustrates a flow diagram of one method of the present invention.

FIG. 12 representatively illustrates a flow diagram of one method of the present invention.

Corresponding reference characters indicate corresponding parts throughout the drawings.

### DETAILED DESCRIPTION OF THE DRAWINGS

### Definitions

Within the context of this specification, each term or phrase below includes the following meaning or meanings:

The terms “disposed on,” “disposed along,” “disposed with,” or “disposed toward” and variations thereof are intended to mean that one element can be integral with another element, or that one element can be a separate structure bonded to or placed with or placed near another element.

“Graphical user interface” refers to a visual display, such as that found on a monitor, computer display, hand-held computer or personal-digital assistant, or other such information device, and includes the ways by which a user sees and manipulates information displayed via the interface. For example, a graphical user interface may include a visual representation of an x-y chart, with the x dimension corresponding to time and the y dimension corresponding to pressure or other parameter that correlates with non-nutritive sucking events. The visual representation can be updated periodically so that the visual representation presents a real-time display of the non-nutritive sucking events detected by a pacifier adapted to detect such events.

“Operatively connected” refers to the communication pathway by which one element, such as a sensor, communicates with another element, such as an information device. Communication may occur by way of an electrical connection through a conductive wire. Or communication may occur via a transmitted signal such as an infrared frequency, a radio frequency, or some other transmitted frequency signal. Alternatively, communication may occur by way of a mechanical connection, such as a hydraulic or pneumatic connection.

These terms may be defined with additional language in the remaining portions of the specification.

### Representative Devices and Sensors for Detecting Non-Nutritive Sucking Events

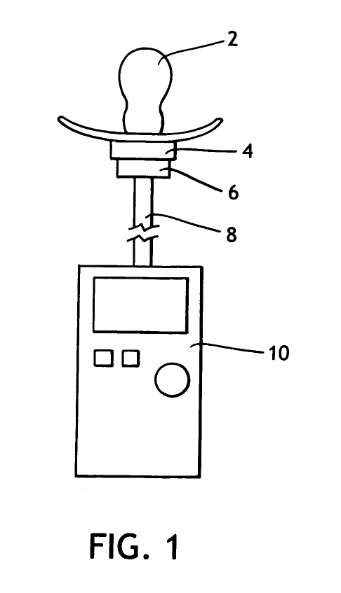

A representative device, adapted to detect non-nutritive sucking events produced by an infant, is depicted in FIG. 1, along with an information device. A nipple 2 is attached to a base 4. A sensor 6 is attached to the base so as to detect an infant sucking on the nipple. For example, as outlined in the Examples section below, commercially available pacifiers were modified (e.g., a NUK®) brand pacifier made by NUK, MAPA GmbH, Industriestrasse 21-25, D-27404, Zeven, Germany; a MAM® brand pacifier made by MAM Babyartikel GEsmbh, Lorenz-Mandl-Gasse 50, 1160 Wien, Austria; a Disney® brand/The-first-years®-brand pacifier made by The First Years, One Kiddie Drive, Avon, Mass.; and a Playtex® brand pacifier, made by Playtex, a business having offices in Allendale, N.J.) by attaching a pressure transducer to the base of each pacifier using an epoxy glue. For a number of pacifiers (e.g., those described in Examples 1-3 below), pressure transducers available from Omega Engineering, having offices at One Omega Drive, Box 4047, Stamford, Conn. were used. As discussed below in Examples 1-3, three different pressure transducers, each capable of measuring different ranges of pressure, were used: (1) 0 to 1 pound per square inch (PSI) (model no. PX26-001 GV, which corresponds to 0 to about 16.7 millivolts DC full scale); (2) 0 to 5 PSI (model no. PX26-005GV, which corresponds to 0 to about 50 millivolts DC full scale); and (3) 0 to 15 PSI (model no. PX26-015GV, which corresponds to 0 to about 100 millivolts DC full scale). An epoxy adhesive available from Cole-Parmer Instrument Company, having offices at 625 East Bunker Court, Vernon Hills, Ill., was used to attach the pressure transducer to the base of the pacifier. By operatively connecting the modified pacifier to a digital multimeter available from Fluke Corporation, having offices at 6920 Seaway Boulevard, Everett, Wash. (or, as described below, a computer having an analog-to-digital device), changes in pressure inside the nipple of the modified pacifier were able to be monitored. In FIG. 1, the operative connection is exemplified by a wire 8 connected to an information device 10, in this case the aforementioned multimeter. As mentioned elsewhere, the sensor 6 may be operatively connected to an information device in various ways, including use of a conductive wire, a selected portion of the electromagnetic spectrum (e.g., a wireless connection using radio waves), or a mechanical connection (e.g., a pneumatic connection).

As described in Example 4 below, some pacifiers were made such that a pressure transducer and wireless transmitter were attached to the base of various pacifier models. As with the pacifiers discussed above, the pressure transducer is in fluid communication with the air within the nipple so that, as described below, changes in pressure due to an infant sucking on the pacifier are detected and wirelessly transmitted to an information device, such as a computer.

An infant's sucking on the nipple causes the flexible nipple to stretch or extend, and then return to its original shape. This periodic extending or stretching of the nipple subjects the inside of the nipple to periodic compression, thereby changing the pressure inside the nipple. By operatively connecting a pressure transducer to the volume inside the nipple, the pressure inside the nipple, or a value corresponding to pressure inside the nipple, can be monitored. In some instances, a multimeter was used to display a reading, in millivolts, that corresponded to the pressure inside the nipple. In other cases a computer with an analog-to-digital device was used in conjunction with software adapted to configure the computer for collecting and processing data. Specifically, the computer and software were used to process and display readings corresponding to the pressure inside the nipple. Additional detail regarding the construction of representative pacifiers adapted to detect non-nutritive sucking events and corresponding rhythmic sucking patterns, and representative versions of systems and other contexts employing such pacifiers, are given elsewhere in the present application, and in the Examples section below.

### EXAMPLE 1

### Construction of One Version of a Device Adapted to Detect Non-Nutritive Sucking Events and/or Rhythmic Sucking Patterns

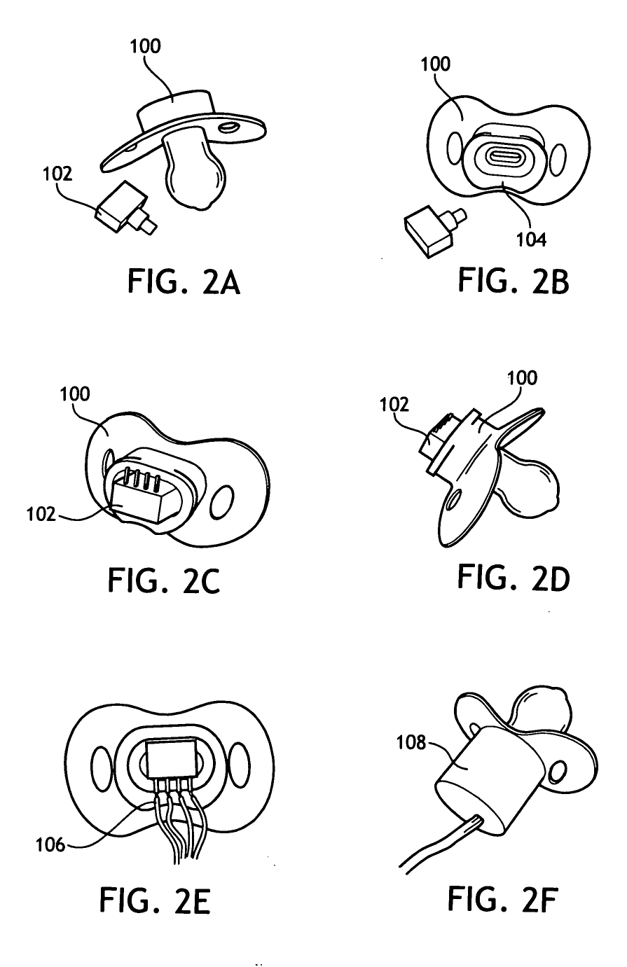

A Disney®-brand pacifier, manufactured by The First Years, One Kiddie Drive, Avon, Mass., was obtained. The pacifier was modified as depicted in FIGS. 2A through 2F. FIG. 2A depicts the pacifier 100 along with a pressure transducer 102 obtained from Omega Engineering, having offices at One Omega Drive, Box 4047, Stamford, Conn. This particular pressure transducer (model no. PX26-001 GV) was adapted to detect pressure readings from zero to one pound per square inch. FIG. 2B depicts the pacifier 100 modified such that the back of the base has been removed to reveal the end of the nipple 104 which is attached to the base.

FIGS. 2C and 2D show the pressure transducer 102 inserted into the opening created by removing the back of the base. In this version of a device adapted to detect non-nutritive sucking events and/or rhythmic sucking patterns, epoxy is used to attach the pressure transducer 102 to the base of the pacifier. A sufficient amount of epoxy was used so that epoxy filled the space between the portion of the transducer that was inserted into the base and the inner wall of the base. A substantially air-tight seal was formed between the transducer and the base of the pacifier.

FIG. 2E shows a four-conductor ribbon cable 106 soldered to the pressure transducer. FIG. 2F depicts a protective sleeve 108 positioned around the pressure transducer. Note also that a drill with a #60 drill bit was used to drill a hole through the solidified epoxy (at a location near the transducer) such that a port to the open end of the nipple was created. The port allowed the nipple to vent air from its interior when squeezed or compressed; and to draw air into its interior when allowed to return to its original shape. The port allowed the nipple to collapse and return to its original shape during use. The port was added because the original, unmodified pacifier had a vent/port, but the original vent was eliminated when the back of the pacifier was removed and the transducer was attached with epoxy.

### EXAMPLE 2

### Construction of Another Version of a Device Adapted to Detect Non-Nutritive Sucking Events and/or Rhythmic Sucking Patterns

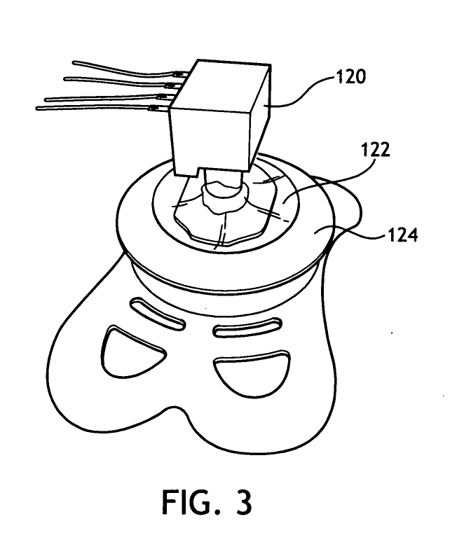

A MAM®-brand pacifier made by MAM Babyartikel GEsmbh, Lorenz-Mandl-Gasse 50, 1160 Wien, Austria, was obtained. The pacifier was modified as follows. As depicted in FIG. 3, a pressure transducer 120, obtained from Omega Engineering, having offices at One Omega Drive, Box 4047, Stamford, Conn., was attached to the open end 122 of a base 124 of a pacifier. Epoxy was used to attach the pressure transducer to the pacifier. A sufficient amount of epoxy was used so that epoxy filled the space between the portion of the transducer that was inserted into the base and the inner wall of the base. A substantially air-tight seal was formed between the transducer and the base of the pacifier. This particular pressure transducer was adapted to detect pressure readings of zero to 5 pounds per square inch. As with Example 1, a #60 drill bit was used to drill a hole into the side of the epoxy such that a port to the open end of the nipple was created. The port allowed the nipple to vent air from its interior when squeezed or compressed; and to draw air into its interior when allowed to return to its original shape.

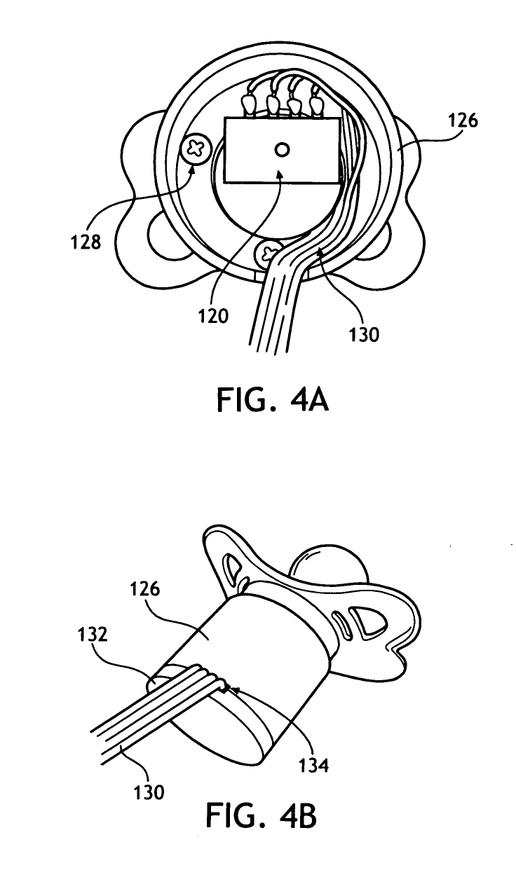

FIG. 4A depicts a protective cup 126 attached to the base of the pacifier using #2-56 screws 128. A four-conductor ribbon cable 130 was soldered to each of the four connectors of the pressure transducer. FIG. 4B shows the modified pacifier with an end cap 132 attached to the protective cup 126. A notch 134 allows for egress of the ribbon cable 130 from the interior of the protective cup 126. Note also that a drill with a #60 drill bit was used to drill a hole through the solidified epoxy (at a location near the transducer) such that a port to the open end of the nipple was created. The port allowed the nipple to vent air from its interior when squeezed or compressed; and to draw air into its interior when allowed to return to its original shape. The port allowed the nipple to collapse and return to its original shape during use. The port was added because the original, unmodified pacifier had a vent/port, but the original vent was eliminated when the back of the pacifier was removed and the transducer with epoxy was attached.

### EXAMPLE 3

### Construction of Another Version of a Device Adapted to Detect Non-Nutritive Sucking Events and/or Rhythmic Sucking Patterns

A Nuk®-brand pacifier, manufactured by NUK, MAPA GmbH, Industriestrasse 21-25, D-27404, Zeven, Germany, was obtained. The pacifier was modified in the same way as the pacifier discussed in Example 2. In this case, however, a pressure transducer capable of detecting 0 to 15 pounds per square inch (0 to 100 millivolt DC, output) was attached to the base of the pacifier. The pressure transducer (model no. PX26-015GV; 0-15 psi) was obtained from Omega Engineering, having offices at One Omega Drive, Box 4047, Stamford, Conn.

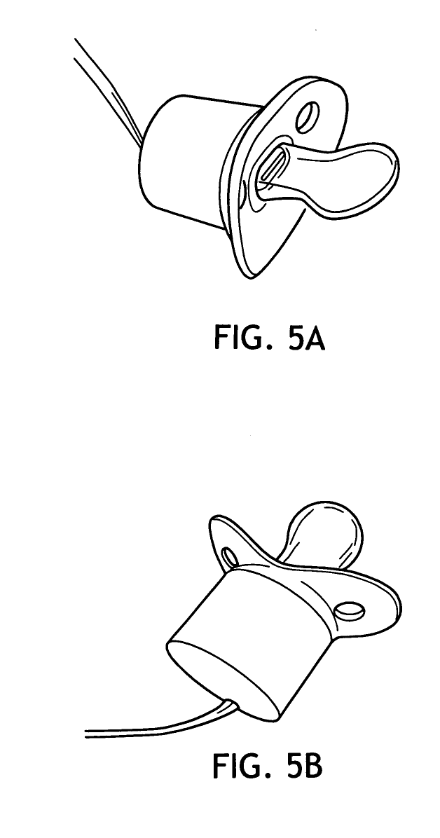

FIGS. 5A and 5B depict two different views of this version of a device adapted to detect non-nutritive sucking events and/or rhythmic sucking patterns.

The previous examples utilized a drilled hole to vent the pacifier. However, subsequent iterations were conducted without venting. These trials resulted in stronger signals and were still accepted by most infants. Therefore, suitable pacifiers may include vented or unvented versions.

### Example 4

### Construction of Another Version of a Device Adapted to Detect Non-Nutritive Sucking Events and/or Rhythmic Sucking Patterns

Several pacifiers were modified to include: a battery; a voltage regulator (model number MIC5219, from Micrel, a business having an office in San Jose, Calif.); a pressure transducer (model number 1451-015 G-T, from MSI Sensors, a business having an office in Hampton, Va.); a radio/microcontroller module (in this case one employing a ZIGBEE brand standard wireless protocol for transmitting or receiving data in digital form) (model number RC2200AT-SPPIO, from Radiocrafts, a business having an office in Sandakerveien 64, NO-0484 Oslo, Norway); an instrumentation amplifier (model number AD627, from Analog Devices, a business having an office in Norwood, Mass.); and an ultra-precision operational amplifier (model number OP177, from Analog Devices, a business having an office in Norwood, Mass.).

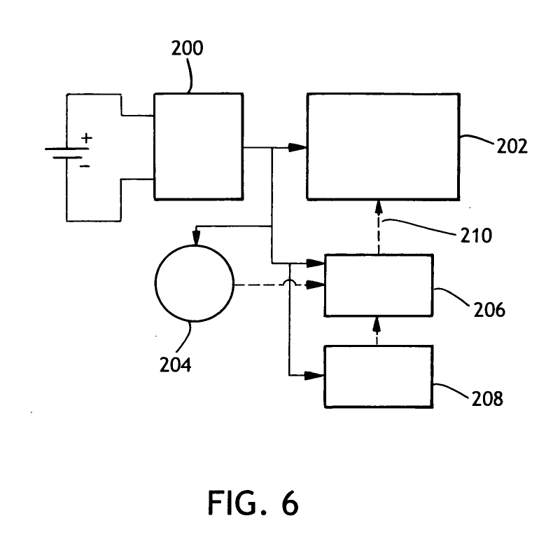

In this representative embodiment, the wireless pacifier was powered by the output of a 3-volt, 500 milli-ampere voltage regulator 200 in FIG. 6. Typically, two 3-volt coin cells configured in series provided the raw unregulated voltage. This was necessary since the regulator needed greater than 3.1 volts to operate. The 3-volt coin cells arranged in series (for a total of 6 volts), as compared to a single 3-volt coin cell, improved the utilization of available batteries.

As noted above, in this representative embodiment of a wireless pacifier, the main component was a ZIGBEE brand ready-radio module, designated as 202 in FIG. 6. This module contained the necessary components for bi-directional wireless communications. The module also contained a microcontroller employing a 10-bit analog-to-digital converter input. The radio and microcontroller worked together to process information and create a wireless serial link between the pacifier and, as discussed below, a computer. The microcontroller engaged the radio at a rate of 10 Hertz to transmit 80 bytes of information, each cycle, for a transmission rate of 800 bytes per second (i.e., a 100 Hertz sampling rate). The 80 bytes of information consisted of 10 packets of 8 bytes each. The binary encoded data was transmitted in hex format (16-byte characters) as follows: byte 0: pacifier wireless module identification; byte 1-3: sample number, with each increment corresponding to 10 milliseconds in time; byte 4-7: pressure in IEEE 754 32-bit format; byte 8: pacifier wireless module identification; byte 9-11: sample number, with each increment corresponding to 10 milliseconds in time; byte 12-15: pressure in IEEE754 32 bit format.

The pressure transducer 204, which in this representative embodiment was capable of detecting in the range of 0 to 15 gauge pounds per square inch, was of a piezo-resistive silicon type. The transducer employed a bridge network that required voltage excitement. The differential pressure output signal was passed to an instrumentation amplifier 206. The instrumentation amplifier was provided a reference voltage from an ultra-precision amplifier 208 which was configured as a voltage follower fed by a simple voltage divider. The instrumentation amplifier then provided the proper level signal to the module's analog-to-digital converter 210 where its output is adjusted to represent the actual output of the pressure transducer in pounds per square inch, or “PSI.”

The wireless pacifier was prepared much like the wired-in pacifiers described in the previous examples, with the exception that the above components, interconnected as depicted in FIG. 6, were placed in the base of the pacifier. Thus the resulting, modified pacifiers looked generally like that depicted in FIGS. 5A and 5B with the exception, of course, of a wire protruding from the base of the pacifier (because the pacifiers transmitted data in wireless fashion). The pacifiers modified as generally described above included two DISNEY brand pacifiers, one for ages zero and over, and one for ages 3 months and over, both of which had a silicon nipple; two MAM brand pacifiers, one designated as “Crystal” for ages 6 months and over, and one designated “Pearl” for ages zero to 6 months, both of which had a silicon nipple; two NUK brand pacifiers, one designated for ages zero to 6 months, and one designated for 7 to 18 months, both of which had a latex nipple; and two PLAYTEX brand pacifiers, one designated for ages 3 months and over and having a silicon nipple, and one for the same age range but having a latex nipple.

Other sensors may be used to detect non-nutritive sucking events produced by an infant. For example, a strain gauge could be attached to a pacifier to detect any deflection or deformation of one or more elements of the pacifier (e.g., the nipple; the base to which the nipple is attached; etc.).

Alternatively, a sensor for detecting electrical signals associated with contraction of a muscle or muscle group could be used to detect non-nutritive sucking events produced by an infant. For example, a sensor comprising electrodes and capable of being adhered to skin could be used to detect such sucking events, whether effected by an infant sucking on a pacifier, the nipple on a bottle, the infant's own thumb, finger, or fingers, and the like.

If the non-nutritive sucking events produce sounds, then a device for monitoring audible events could be used to detect the sounds corresponding to non-nutritive sucking events.

While the preceding paragraphs provide examples of devices, sensors, and methods that may be used to detect non-nutritive sucking events produced by an infant, as well as different ways by which infants effect non-nutritive sucking events (e.g., by sucking on a pacifier, a nipple on a bottle, the infant's own thumb, etc.), other devices, sensors, methods, and ways of generating non-nutritive sucking events may be used, so long as the selected approach is capable of detecting the non-nutritive sucking events.

### Representative Information Devices

A multimeter, discussed in the preceding paragraphs and in the Examples section below, is one version of an information device; i.e., a device adapted to accomplish one or more of receiving, storing, processing, displaying, or transmitting information, in this case information corresponding to non-nutritive sucking events produced by an infant, and detected by a sensor. The multimeter was used in some cases to display a reading, in millivolts, that corresponded to the pressure inside a modified pacifier. An infant's sucking on that pacifier produced a measurable rhythmic sucking pattern (“RSP”). The RSP was recorded as a series of millivolt readings that could be plotted and evaluated.

A variety of information devices may be used in conjunction with the present invention. For example, a computer may be used to monitor one or more values corresponding to the non-nutritive sucking events produced by an infant. Generally, a computer is capable of receiving, storing, processing, displaying, and transmitting information. Through the use of appropriate software, the computer can be configured to receive, store, process, display, and/or transmit information corresponding to non-nutritive sucking events produced by an infant. A computer was used to accumulate individual millivolt readings corresponding to individual non-nutritive sucking events. These readings were processed further to calculate an average value for a target infant under various conditions. This work is discussed in more detail below, but it is highlighted now to provide an example of how one version of an information device is used to quantify and process non-nutritive sucking events and rhythmic sucking patterns, which can provide a basis for comparison to other sucking patterns obtain under different conditions and/or obtained from different subjects.

Many different information devices may be used with the present invention. In addition to a desktop computer or a device for recording and/or displaying readings corresponding to non-nutritive sucking events (e.g., a multimeter displaying millivolt readings), one could use a personal-digital assistant, hand-held computer, a portable computer, or other compact device to receive, store, process, display, and/or transmit information corresponding to non-nutritive sucking events produced by an infant. Alternatively, a chart recorder or other such device for recording the detected non-nutritive sucking events may be used. As noted above, the information device may comprise a storage device, including, for example, RAM (i.e., Random Access Memory), ROM (i.e., Read-Only Memory), EPROM (i.e., Erasable Programmable Read-Only Memory), PROM (i.e., Programmable Read-Only Memory), RFID (i.e., Radio Frequency IDentification), or the like. Furthermore, information devices comprising storage devices such as those identified in the preceding list may be compact enough to be attached to the sensor used to detect non-nutritive sucking events produced by an infant. For example, an RFID device could be incorporated into a pacifier such that the device recorded the non-nutritive sucking events produced by an infant sucking on the nipple of the pacifier. When desired, an external device could be used to read the stored information on the RFID element. Alternatively, the information on the RFID element could be viewed on a display, either on the pacifier itself, or on a computer or other information device remote from the pacifier. In some versions of the invention, the information device will comprise only a storage device.

In some versions of the invention, a display connected to the sensor itself (e.g. a pacifier having both a pressure transducer to sense the non-nutritive sucking events and an information device comprising a display) could provide an indication or information corresponding to non-nutritive sucking events detected by the sensor and/or stored on the information device. In some examples of the invention, the display could be graphical in nature, with displayed images corresponding to a condition indicated by non-nutritive sucking events and/or rhythmic sucking patterns. Furthermore, color might be used, either alone, or in conjunction with graphical images, to convey information regarding the condition of the infant. Any hardware and software capable of presenting a display of information, graphical or otherwise, might be used, including, for example, liquid-crystal displays, light-emitting diodes, and the like. In some versions of the invention, the graphical display is associated with an information device remote from the sensor used to detect non-nutritive sucking events and/or rhythmic sucking patterns (as is discussed in more detail in the following paragraph).

It should be noted that the information device need only be operatively connected to the sensor used to detect non-nutritive sucking events. Accordingly, the information device might be attached to the sensor itself. Or the information device might be at a location remote from the sensor, with information conveyed by an appropriate wavelength in the electromagnetic spectrum (e.g., radio waves); a conductive wire; or some mechanical connection (e.g., a pneumatic or hydraulic connection). In some cases, the information device may comprise one or more components attached to the sensor used to detect non-nutritive sucking events, and one or more components at a location remote from the sensor.

### Representative Methods for Assessing a Condition

As discussed above, the present invention is generally directed to systems, devices, and methods for detecting non-nutritive sucking events produced by an infant. One aspect of the present invention is a method that utilizes various aspects of sucking patterns to suggest the presence or magnitude of a condition in an infant. For example, the present invention may utilize sucking patterns to suggest the presence of colic in an infant. In another example, the present invention may utilize sucking patterns to assess the level of cognitive awareness presented by an infant. These assessments can include evaluating the stability of the sucking pattern and the duration of time actually in a rhythmic sucking pattern. Additionally or alternatively, these assessments may be accomplished by comparing the sucking pattern of an infant with a comparative pattern. The sucking patterns described herein may be acquired by any suitable method including those disclosed herein.

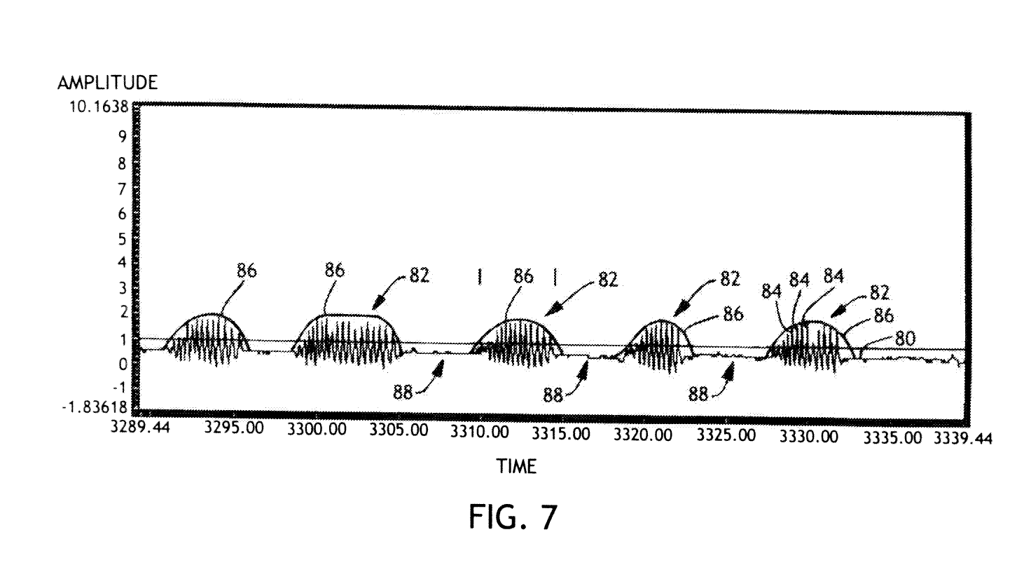

In general, non-nutritive sucking patterns may include rhythmic portions and non-rhythmic portions. The rhythmic portions or rhythmic sucking patterns (RSP) are believed to correlate with an infant's comfort/emotional state. Preliminary observations suggest that healthy, comfortable babies generally express a rhythmic sucking pattern. Additionally, said babies have been observed returning to a rhythmic sucking pattern relatively quickly after being startled, upset, or otherwise disturbed. For example, FIG. 7 is illustrative of a rhythmic pattern associated with a healthy and comfortable baby. FIG. 7 illustrates a first sucking pattern 80 on an X-Y plot with time (in seconds) plotted on the X axis and amplitude (in pounds per square inch, PSI) plotted on the Y axis. A portion of the first sucking pattern 80 includes a plurality of sucking bursts 82. The sucking bursts 82 include a plurality of individual sucks 84. The sucking bursts 82 define a series of envelopes 86. The sucking bursts 82 are generally separated by pauses 88 characterized as periods of relatively little to no sucking. As used herein, the term “envelope” refers to a curve generally defined by the peaks of the individual sucks 84 over the duration of the sucking bursts 82. As illustrated in FIG. 7, the envelopes 86 are illustrated as a line drawn along the peaks of the individual sucks 84. Based on the amplitude of the individual sucks 84 and the duration of the sucking bursts 82, the envelopes define a curve of distinct shape and size.

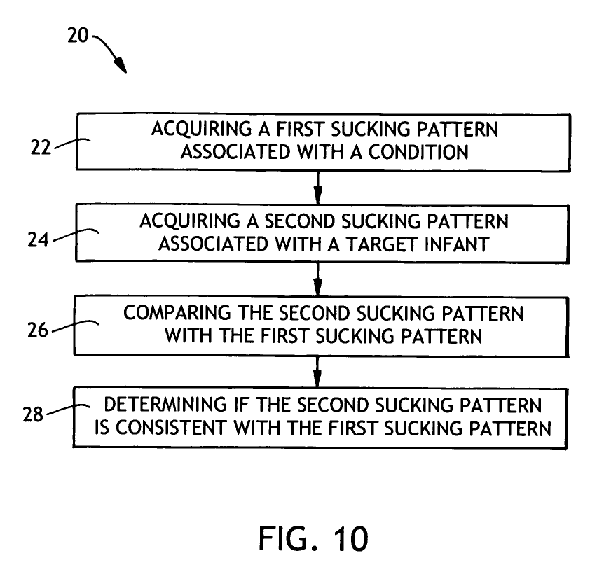

The sucking pattern 80 of FIG. 7 displays several characteristics commonly associated with rhythmic sucking. For example, the amplitudes of the various sucks 84 are relatively consistent at about 2 PSI. Additionally, the various sucking bursts 82 are relatively consistent in duration and are relatively consistent in the number of individual sucks 84 per burst 82. In this example, the sucking bursts 82 have a duration of about 5 seconds and most sucking bursts 82 include about 13 individual sucks 84. Finally, the pauses 88 are relatively consistent in duration. Specifically, in the illustrated sucking pattern 80 of FIG. 10, the pauses 88 have a duration of about 5 seconds.

In contrast, preliminary observations suggest that infants with certain conditions, such as colic, do not express a typical rhythmic sucking pattern or do not express a typical rhythmic pattern as frequently as a typical comfortable infant. Additionally, preliminary observations also suggest that infants with certain conditions, such as colic, require an atypically long period of time to establish a rhythmic pattern as compared to a typical comfortable infant.

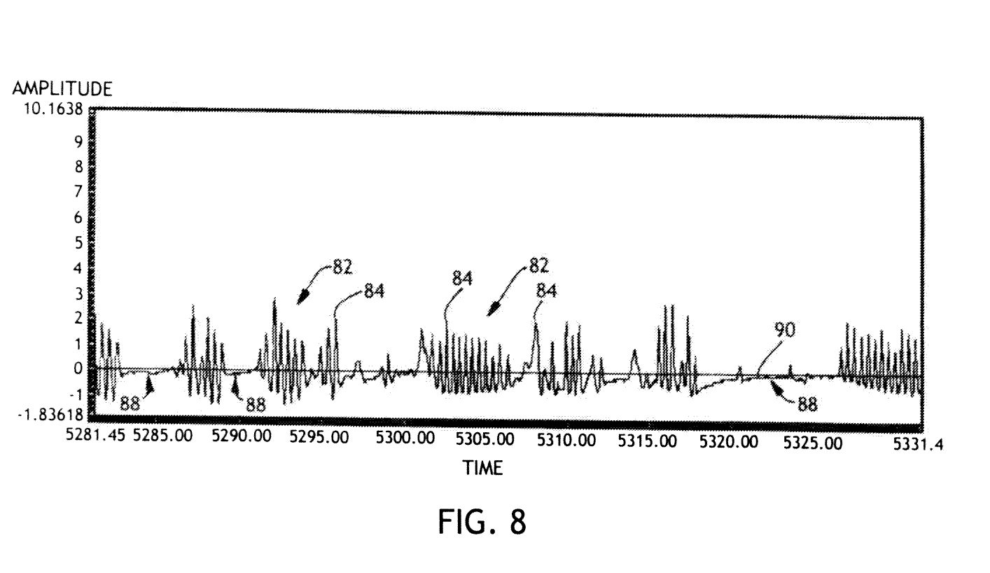

For example, FIG. 8 is illustrative of a non-rhythmic pattern associated with a colicky baby. FIG. 8 illustrates a second sucking pattern 90 on an X-Y plot with time plotted on the X axis and amplitude on the Y axis. This portion of the second sucking pattern 90 includes a plurality of individual sucks 84 but does not present well defined sucking bursts 82 or envelopes 86.

The second sucking pattern 90 of FIG. 8 displays several characteristics commonly associated with non-rhythmic sucking. For example, the amplitudes of the individual sucks 84 are relatively inconsistent and range from about 0.5 PSI to about 3 PSI. Additionally, the sucking bursts 82 are difficult to identify and are relatively inconsistent in duration. For example, the sucking bursts 82 have a duration varying from about 3 seconds to about 7 seconds. Additionally, the numbers of individual sucks 84 per burst 82 are relatively inconsistent. For example, the numbers of individual sucks 84 per burst 82 vary from about 7 to 13. Finally, the pauses 88 are difficult to identify and are relatively inconsistent in duration.

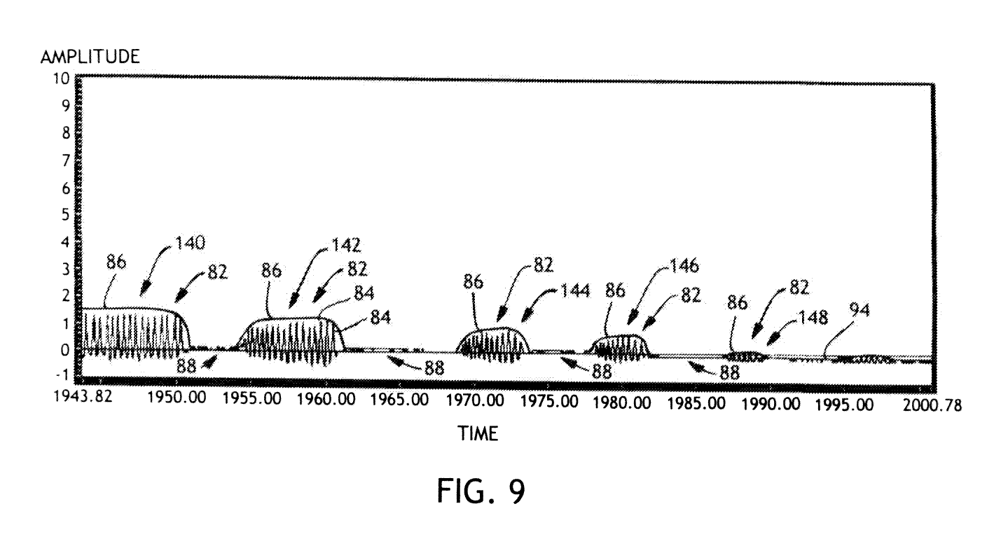

Preliminary observations also suggest that healthy, comfortable babies generally express a rhythmic sucking pattern having decreasing amplitude as they fall asleep. It is also believed that this pattern reflects a decreasing cognitive awareness. For example, FIG. 9 is illustrative of a rhythmic pattern associated with a healthy and comfortable baby falling asleep. FIG. 9 illustrates a third sucking pattern 94 on an X-Y plot with time (in seconds) plotted on the X axis and amplitude (in pounds per square inch, PSI) plotted on the Y axis. A portion of the third sucking pattern 94 includes a plurality of sucking bursts 82. The sucking bursts 82 include a plurality of individual sucks 84. The sucking bursts 82 define a series of envelopes 86. The sucking bursts 82 are generally separated by pauses 88 characterized as periods of relatively little to no sucking. As illustrated in FIG. 7, the envelopes 86 are illustrated as a line drawn along the peaks of the individual sucks 84. Based on the amplitude of the individual sucks 84 and the duration of the sucking bursts 82, the envelopes define a curve of distinct shape and size.

The sucking pattern 94 of FIG. 9 illustrates a first sucking burst 140, a second sucking burst 142, a third sucking burst 144, a fourth sucking burst 146, and a fifth sucking burst 148. The sucking bursts 140-148 have declining amplitudes of about 1.4 PSI, 1 PSI, 0.7 PSI, 0.5 PSI, and 0.2 PSI, respectively. This pattern was recorded as the subject infant was transitioning into a light sleep where sucking continues. When the infant reaches a deep sleep the sucking ceases and the pattern changes to essentially a flat line. Thus, it is believed that the sucking pattern 94 is illustrative of declining cognitive awareness. Also, it is believed that sucking patterns may be used to estimate the relative depth of sleep of a target infant.

Those skilled in the art will readily appreciate that having a method of determining an infant's state of cognitive awareness and/or depth of sleep may be useful for various reasons. For example, it may be advantageous to perform a procedure on an infant when the infant is exhibiting low cognitive awareness. For example, the heel prick test may be less traumatic if administered to newborn infants when the infant is in a state of low cognitive awareness. Likewise, removing adhesive pads attached to an infant may be easier if administered when the infant is in a state of low cognitive awareness.

In one aspect, the present invention provides a method of suggesting the presence or degree of a condition in an infant. The method 20 is schematically illustrated in FIG. 10. The method 20 includes a first step 22 of acquiring a first sucking pattern associated with the condition; a second step 24 of acquiring a second sucking pattern associated with a target infant; a third step 26 of comparing the second sucking pattern with the first sucking pattern; and a fourth step 28 of determining if the second sucking pattern is consistent with the first sucking pattern.

The first step 22 is acquiring a first sucking pattern associated with the condition. As used herein, the term “sucking pattern associated with the condition” refers to a typical sucking pattern expressed by an infant having the condition. For example, a pattern obtained from an infant having colic results is a sucking pattern “associated with” the colic condition. Likewise, a pattern obtained from one or more infants progressing in stages of sleep results in sucking patterns associated with varying degrees of cognitive awareness.

The first sucking pattern, which is associated with a condition, may be based on any suitable population and may be acquired in any suitable manner. For example, the first sucking pattern may be acquired by studying one or more infants currently presenting a given condition. The various patterns associated with the condition may be utilized individually or may be aggregated in any suitable manner. For example, the various patterns may be aggregated from two or more infants presenting the given condition.

Aggregation may be achieved by any suitable means. For example, aggregation may include averaging the amplitude of the individual sucks, averaging the envelope function, averaging the number of individual sucks per sucking burst, averaging the ratio of duration sucking relative to the duration of pausing, or the like, or combinations thereof. Any suitable software may be used to aggregate one or more aspects of one or more sucking patterns. For example, software utilizing artificial intelligence may be used to average various data and/or look for trends and patterns.

In some embodiments, a first pattern associated with colic may be acquired from one or more infants diagnosed as having colic and presenting a colicky condition. It is believed that the sucking pattern associated with colicky infants exhibits a larger percentage of non-rhythmic patterns as illustrated in FIG. 8.

As discussed above, FIG. 8 displays several characteristics commonly associated with non-rhythmic sucking. For example, the amplitudes of the individual sucks 84 are relatively inconsistent. Additionally, the sucking bursts 82 are difficult to identify and are relatively inconsistent in duration. Additionally, the numbers of individual sucks 84 per burst 82 are relatively inconsistent. Finally, the pauses 88 are difficult to identify and are relatively inconsistent in duration.

Also as discussed above, FIG. 9 displays characteristics commonly associated with infants in varying degrees of cognitive awareness. It is believed that the sucking pattern associated with infants in varying degrees of cognitive awareness exhibit rhythmic patterns with declining amplitude.

The second step 24 in the method 20 is acquiring a second sucking pattern associated with a target infant. The second sucking pattern associated with a target infant may be based on any suitable duration of time and may be acquired in any suitable manner. For example, the second sucking pattern may be acquired from the target infant on a single occasion. Alternatively, the second sucking pattern may be aggregated from the target infant on more than one occasion. The method of aggregating the second sucking pattern may be accomplished in any suitable manner as discussed above. In some embodiments, the first step 22 may be completed before the second step 24. In some embodiments, the second step 24 may be completed before the first step 22. In some embodiments, the first step 22 and the second step 24 may be completed at 10 relatively the same time.

When acquiring the second sucking pattern, it is desirable to reproduce the conditions present during the acquisition of the first sucking pattern to minimize other variables affecting the assessment. In other words, efforts may be taken to minimize subjective effects. For example, it may be desirable to use the same type of pacifier, the same room/environment, and the same administrator. Additionally, it may be desirable for the feeding conditions of the infant to be similar. For example, if the baby was recently fed before acquiring the first pattern, then it may be desirable for the baby to be similarly fed before acquiring the second pattern. Likewise, the time of the day may be reproduced to be as consistent as possible.

The third step 26 of the method 20 includes comparing one or more aspects of the second sucking pattern with one or more aspects of the first sucking pattern. In various embodiments, the comparison step may be a subjective evaluation performed by a trained technician and/or the comparison may be an objective evaluation based, at least in part, on pattern recognition software.

The fourth step 28 of the method 20 includes determining if the second sucking pattern associated with the target infant is consistent with the first sucking pattern associated with the condition. The determination step may be accomplished by subjective evaluation or by utilizing any suitable pattern analyzing software. In some embodiments, the pattern analyzing software may include algorithms based on Signal Detection Theory, Hidden Markov models, or neural network analysis, and the like, and combinations thereof. The pattern analyzing software may be used to measure/characterize various aspects of the patterns such as, for example, the shape/function of the envelope 86, the duration of the sucking bursts 82, the amplitude of the individual sucks 84, the frequency of the individual sucks 84, the duration of the pauses 88, the number of individual sucks 84 per sucking burst 82, and the like, and combinations thereof.

While it is preferred to hold all variables constant, as discussed above, in some embodiments, the first sucking pattern and/or the second sucking pattern may be obtained in various locations and at various times. For example, the first sucking pattern and/or the second sucking pattern may be obtained in the infant's home, in a clinic, or any other suitable location. In various embodiments, the comparing step and/or the determining step may be performed in any suitable location and any suitable time. In one embodiment, the second sucking pattern is acquired in a clinic and the comparison is performed in a clinic.

In some embodiments, the second sucking pattern may be acquired in a first location and the comparing step may be performed at a second location different than the first location. In some embodiments, the first location may be the infant's residence and the second location may be a clinic.

### Representative Approaches to Monitoring the Progress of Treatment

The present invention also includes a method to monitor progress in the treatment of a target infant having a condition. It is difficult to detect improvement in certain conditions. For example, current methods of monitoring the progress of colic treatment include determining the number of hours the infant screams or cries. This requires extensive monitoring and long durations of crying. The present invention may be adapted to measure the percentage of time an infant presents a rhythmic sucking pattern versus the time an infant presents a non-rhythmic sucking pattern. An increase or decrease in the percentage of time in a rhythmic pattern is believed to be indicative of an improved condition or a worsened condition respectively. Thus, it is believed that a quantitative measure of an infant's progress can be documented over an extended period of time.

In one embodiment, the present invention provides a simple feedback system wherein a caregiver may assess an infant's state of colic. With this method, different treatments can be directly assessed for efficacy in calming a colic child. Additionally, the present invention can record the duration of time an infant is in a rhythmic sucking pattern as a percentage of time. It is believed that this method will allow a caregiver to assess patterns and determine what is working and what is not. Additionally, it is believed that this method will improve the ability to observe small changes over extended periods of time.

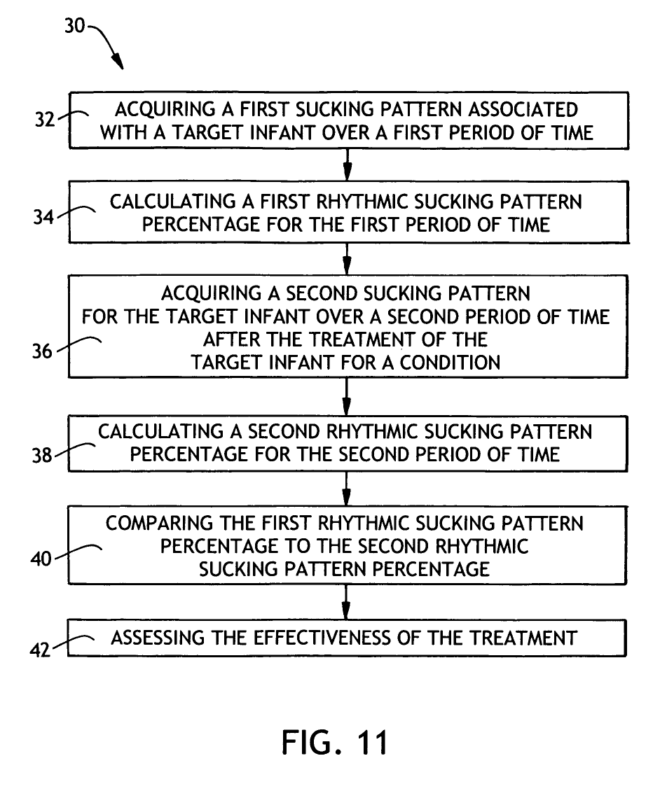

In one aspect, a method 30 of monitoring progress of a treatment of a target infant having a condition is representatively illustrated in FIG. 11. The method 30 may include a first step 32 of acquiring a first sucking pattern associated with the target infant over a first period of time; a second step 34 of calculating a first rhythmic sucking pattern percentage for the first period of time; a third step 36 of acquiring a second sucking pattern for the target infant over a second period of time after the treatment of the target infant for the condition; a fourth step 38 of calculating a second rhythmic sucking pattern percentage for the second period of time; a fifth step 40 of comparing the first rhythmic sucking pattern percentage to the second rhythmic sucking pattern percentage; and a sixth step 42 of assessing the effectiveness of the treatment.

As discussed above, the first step 32 of acquiring a first sucking pattern for a target infant over a first period of time may be accomplished by any suitable means. For example, the representative devices and sensors for detecting non-nutritive sucking events may be used to obtain the first sucking pattern. The first period of time may be of any suitable duration. For example, in some embodiments, the first period of time may be 10 minutes, 1 hour, 2 hours, 5 hours, 24 hours, or more.

The second step 34 includes calculating a first rhythmic sucking pattern percentage for the first period of time. The first rhythmic sucking pattern percentage is calculated by dividing the time the infant maintains a rhythmic sucking pattern in the first time period by the total of the first period of time. The rhythmic sucking pattern percentage may be determined by subjective observation, pattern recognition software, or other automated software/systems.

The third step 36 includes acquiring a second sucking pattern for the target infant over a second period of time. The second period of time occurs after a course of treatment of the target infant for the condition. The second period of time may be of any suitable duration. In some embodiments the second period of time may be dependent upon the type of treatment being administered. In general, the treatment should be given time to have some effect prior to acquiring the second sucking pattern. Also, as discussed above, it may be desirable to reproduce the conditions present during the acquisition of the first sucking pattern when acquiring the second sucking pattern to minimize other variables affecting the assessment.

The fourth step 38 includes calculating a second rhythmic sucking pattern percentage for the second period of time. The second rhythmic sucking pattern percentage is calculated by dividing the time the infant maintains a rhythmic sucking pattern in the second time period by the total of the second period of time. The rhythmic sucking pattern percentage may be determined by subjective observation, by pattern recognition software, or other suitable automated software/systems.

The fifth step 40 includes comparing the first rhythmic sucking pattern percentage to the second rhythmic sucking pattern percentage. The comparison is simply the relative percentages for each time period. The comparing step 40 may be accomplished by a technician, by computer software, or combinations thereof.

The sixth step 42 includes assessing the effectiveness of the treatment. For example, if the second rhythmic sucking pattern percentage is greater than the first rhythmic sucking pattern percentage then there is some suggestion that the given treatment had some effect. Likewise, if the second rhythmic sucking pattern percentage is less than or equal to the first rhythmic sucking pattern percentage then there is some suggestion that the given treatment was not effective.

In some embodiments, the method 30 may further include the step of acquiring a third sucking pattern for the target infant over a third period of time after a second treatment of the target infant for the condition, wherein the second treatment is different than the first treatment. In these embodiments, the method includes calculating a third rhythmic sucking pattern percentage for the third period of time and comparing the third rhythmic sucking pattern percentage to the second rhythmic sucking pattern percentage and/or the first rhythmic sucking pattern percentage to determine the effectiveness of the first treatment relative to the second treatment.

In some embodiments, at least one of the first sucking pattern, second sucking pattern, or third sucking pattern are electronically stored and transmitted to a medical professional by any suitable means.

### Representative Approaches to Monitoring the Progress of Treatment Relative to a Population

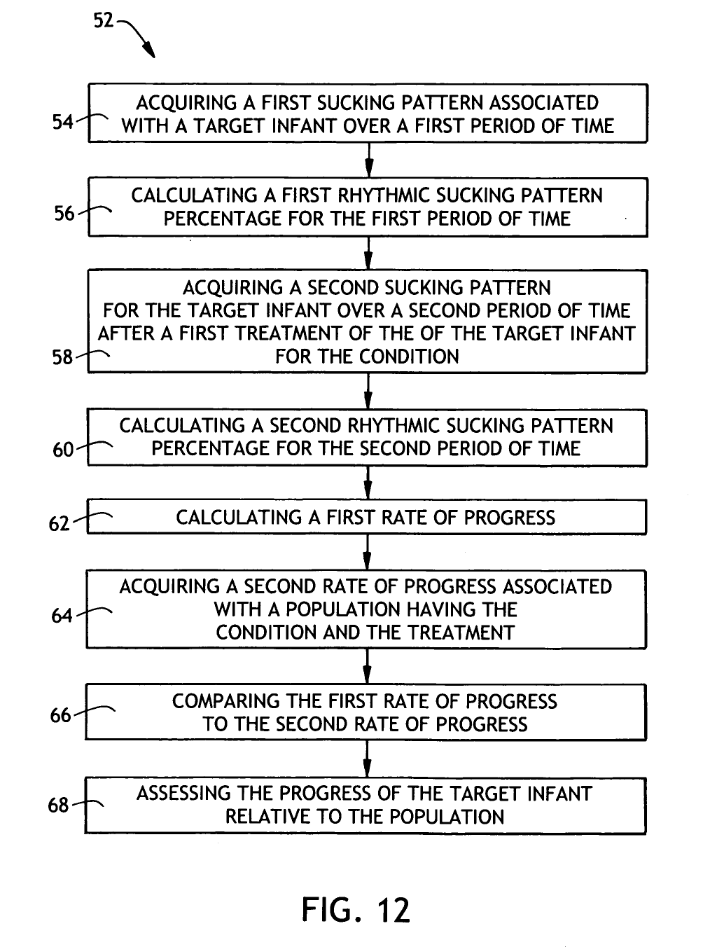

The present invention also provides a method for monitoring the progress of a treatment (at a point in time) of a target infant having a condition as compared with a select population (at the same point in time) having the same condition and the same treatment. The method 52 is representatively illustrated in FIG. 12. The method 52 includes a first step 54 of acquiring a first sucking pattern for a target infant over a first period of time; a second step 56 of calculating a first rhythmic sucking pattern percentage for the first period of time; a third step 58 of acquiring a second sucking pattern for the target infant over a second period of time after a first treatment of the target infant for the condition; a fourth step 60 of calculating a second rhythmic sucking pattern percentage for the second period of time; a fifth step 62 of calculating a first rate of progress for the target infant at a first point in time; a sixth step 64 of acquiring a second rate of progress as a function of time from a population having the condition and the treatment; a seventh step 66 of comparing the first rate of progress to the second rate of progress at the first point in time; and an eighth step 68 of assessing the progress of the first infant relative to the population at the first point in time. In some embodiments, the steps 54-62 may be started or completed before the start or completion of the steps 64-68. In some embodiments, the steps 54-62 may be started or completed after the start or completion of the steps 64-68. In some embodiments, the steps 54-62 may be started or completed at relatively the same time as the start or completion of the steps 64-68.

The first step 54 includes acquiring a first sucking pattern for a target infant over a first period of time. The first sucking pattern may be acquired in any suitable manner such as those described herein. The first period of time may be of any suitable duration. In some embodiments, the first period of time may be 10 minutes, 1 hour, 2 hours, 5 hours, 24 hours, or more.

The second step 56 includes calculating a first rhythmic sucking pattern percentage for the first period of time. The first rhythmic sucking pattern percentage is calculated by dividing the time the infant maintains a rhythmic sucking pattern in the first time period by the total of the first period of time. The rhythmic sucking pattern percentage may be determined by any suitable means such as the means described above.

The third step 58 includes acquiring a second sucking pattern for the target infant over a second period of time after a first treatment of the target infant for the condition. The second sucking pattern for a target infant may be acquired in any suitable manner, such as those described herein. The second period of time may be any suitable duration. In some embodiments, the second period of time may be 10 minutes, 1 hour, 2 hours, 5 hours, 24 hours, or more.

As discussed above, the treatment should be given time to have some effect. Also, as discussed above, it is desirable to reproduce the conditions present during the acquisition of the first sucking pattern when acquiring the second sucking pattern to minimize other variable affecting the assessment.

The fourth step 60 includes calculating a second rhythmic sucking pattern percentage for the second period of time. The second rhythmic sucking pattern percentage is calculated by dividing the time the infant maintains a rhythmic sucking pattern in the second time period by the total of the second period of time. The rhythmic sucking pattern percentage may be determined by subjective observation, pattern recognition software, or by other suitable automated software/systems.

The fifth step 62 includes calculating a first rate of progress for the target infant at a first point in time. The rate of progress is the difference between the second rhythmic sucking pattern percentage and the first rhythmic sucking pattern percentage over the time from the start of treatment to the acquisition of the second pattern.

The sixth step 64 includes acquiring a second rate of progress as a function of time from a population having the same condition and the same treatment. In some embodiments, the population would be as similar to the target infant as practical. For example, the target infant and the population would preferably have similar ages, dosing, and condition severity. The seventh step 66 includes comparing the first rate of progress at the first point in time to the second rate of progress at the first point in time. The eighth step 68 includes assessing the progress of the first infant relative to the population.

In some embodiments, the second rate of progress may be an aggregate rate of progress as a function of time from a population of infants known to have the same condition and the same treatment.

In the various embodiments and examples discussed herein, the first sucking pattern and/or the second sucking pattern and/or third sucking pattern may be obtained in various locations and in various times. For example, the first sucking pattern and/or the second sucking pattern and/or the third sucking pattern may be obtained in the infant's home, in a clinic, or any other suitable location. In various embodiments, the comparing step and/or the determining step may be performed in any suitable location and any suitable time. In various embodiments, the first sucking pattern and/or second sucking pattern and/or third sucking pattern may be acquired in a clinic and the comparison may be performed in a clinic.

In some embodiments, the second sucking pattern may be acquired in a first location and the comparing step may be performed at a second location different than the first location. In some embodiments, the first location may be the infant's residence and the second location may be a clinic.

In various embodiments, the various components of rhythmic sucking may be monitored, tracked, and calculated, etc., individually or in combination. As discussed above, the components of rhythmic sucking include: intensity, frequency, pauses, number of sucks per envelope, envelope function, and the like. Thus, in various embodiments, any one or more of these components may be measured and assessed in determining if the pattern is “rhythmic.”

While the invention has been described in detail with respect to specific embodiments thereof, it will be appreciated that those skilled in the art, upon attaining understanding of the foregoing will readily appreciate alterations to, variations of, and equivalents to, these embodiments. Accordingly, the scope of the present invention should be assessed as that of the appended claims and any equivalents thereto. Additionally, all combinations and/or sub-combinations of the disclosed embodiments, ranges, examples, and alternatives are also contemplated.

1. A method of assessing a condition in a target infant comprising,
acquiring a first sucking pattern associated with the condition;
acquiring a second sucking pattern for the target infant;
comparing the second sucking pattern with the first sucking pattern; and
determining if the second sucking pattern is consistent with the first sucking pattern.

2. The method of claim 1 wherein the first sucking pattern is an aggregate pattern derived from a population of infants expressing the condition.

3. The method of claim 1 wherein the second sucking pattern is an aggregate pattern derived from the target infant on two or more occasions.

4. The method of claim 1 wherein at least one of the first and the second sucking pattern is acquired with a pacifier adapted to measure at least one of intensity, frequency, pauses, number of sucks per envelope, or envelope function of the sucking patterns.

5. The method of claim 4 wherein the second sucking pattern is acquired in the target infant's residence and the comparing step and the determining steps are performed by a computer adapted to receive data from the pacifier.

6. The method of claim 1 wherein the condition is colic or cognitive alertness.

7. The method of claim 1 wherein the determining step is a subjective evaluation performed by a trained technician.

8. The method of claim 1 wherein the determining step is an objective evaluation based, at least in part, on pattern analyzing software, artificial intelligence, or neural networks.

9. The method of claim 1 wherein the second sucking pattern is acquired in a clinic and the comparison is performed in a clinic.

10. The method of claim 1 wherein the second sucking pattern is acquired in a first location and the comparing step is performed at a second location different than the first location.

11. The method of claim 10 wherein the first location is the infant's residence and wherein the second location is a clinic.

12. A method of monitoring progress of a treatment of a target infant having a condition comprising,
acquiring a first sucking pattern for the target infant over a first period of time;
calculating a first rhythmic sucking pattern percentage for the first period of time;
acquiring a second sucking pattern for the target infant over a second period of time after the treatment of the target infant for the condition;
calculating a second rhythmic sucking pattern percentage for the second period of time;
comparing the first rhythmic sucking pattern percentage to the second rhythmic sucking pattern percentage; and
assessing the effectiveness of the treatment.

13. The method of claim 12 further comprising
acquiring a third sucking pattern for the target infant over a third period of time after a second treatment of the target infant for the condition, wherein the second treatment is different than the first treatment,
calculating a third rhythmic sucking pattern percentage for the third period of time;
comparing the third rhythmic sucking pattern percentage to the second rhythmic sucking pattern or the first rhythmic sucking pattern; and
determining the effectiveness of the first treatment relative to the second treatment.

14. The method of claim 12 wherein at least one of the first sucking pattern, second sucking pattern, or third sucking pattern are electronically stored and electronically transmitted to a medical professional.

15. The method of claim 12 wherein the condition is colic or cognitive alertness.

16. A method of monitoring progress of a treatment of a target infant having a condition comprising,
acquiring a first sucking pattern for the target infant over a first period of time;
calculating a first rhythmic sucking pattern percentage for the first period of time;
acquiring a second sucking pattern for the target infant over a second period of time after a first treatment of the target infant for the condition;
calculating a second rhythmic sucking pattern percentage for the second period of time;
calculating a first rate of progress for the target infant at a first point in time;
acquiring a second rate of progress as a function of time from a population having the condition and the treatment;
comparing the first rate of progress to the second rate of progress at the first point in time; and
assessing the progress of the first infant relative to the population at the first point in time.

17. The method of claim 16 wherein the second rate of progress is an aggregate rate of progress as a function of time from a population of infants known to have the condition and the treatment.

18. The method of claim 16 wherein the condition is colic.

19. The method of claim 16 wherein the first sucking pattern is acquired with a pacifier adapted to measure at least one of intensity, frequency, pauses, number of sucks per envelope, or envelope function of the first sucking pattern.

20. The method of claim 16 wherein the comparing or assessing steps are a subjective evaluation performed by a trained technician, an objective evaluation based in part on pattern analyzing software, or combinations thereof.
